(19) United States
(12) Patent Application Publication
(10) Pub. No.: US 2025/0009950 A1
(43) Pub. Date: Jan. 9, 2025

(54) PERITONEAL DIALYSIS SYSTEM INCLUDING A PATIENT LINE FILTER HAVING A TUBULAR MEMBRANE

(71) Applicants: BAXTER INTERNATIONAL INC., Deerfield, IL (US); BAXTER HEALTHCARE SA, Glattpark (Opfikon) (CH)

(72) Inventors: Steffen WAGNER, Deerfield, IL (US); Ralf FLIEG, Deerfield, IL (US); Reinhold BUCK, Deerfield, IL (US); Christof BECK, Deerfield, IL (US); Rainer BLICKLE, Deerfield, IL (US); Bernd KRAUSE, Deerfield, IL (US); Torsten KNOER, Deerfield, IL (US)

(21) Appl. No.: 18/709,961

(22) PCT Filed: Nov. 18, 2022
(86) PCT No.: PCT/US2022/080123

§ 371 Date: May 14, 2024

Related U.S. Application Data
(60) Provisional application No. 63/291,029, filed on Dec. 17, 2021.

(51) Int. Cl. A61M 1/28; A61M 1/16

(52) U.S. Cl. CPC A61M 1/285; A61M 1/1621

(57) ABSTRACT

A peritoneal dialysis (“PD”) system (10) includes a PD machine (20); a patient line (50) extending from the PD machine (20); and a filter set (100) including a filter housing (102) having a tubular filter membrane (120, such as a sterilizing grade or bacteria reduction filter membrane) positioned and arranged to filter fresh PD fluid flowing radially across the tubular filter membrane (120), and a transfer set-side port (106p) positioned and arranged to receive (i) filtered fresh PD fluid during a patient fill and (ii) used PD fluid during a patient drain.

### PRIORITY CLAIM

The present application claims priority to and the benefit of U.S. Provisional Application No. 63/291,029, filed on Dec. 17, 2021, the entire contents of which are hereby incorporated by reference.

### BACKGROUND

The present disclosure relates generally to medical fluid treatments and in particular to the filtering of treatment fluid during dialysis fluid treatments.

Due to various causes, a person's renal system can fail. Renal failure produces several physiological derangements. It is no longer possible to balance water and minerals or to excrete daily metabolic load. Toxic end products of metabolism, such as, urea, creatinine, uric acid and others, may accumulate in a patient's blood and tissue.

Reduced kidney function and, above all, kidney failure is treated with dialysis. Dialysis removes waste, toxins and excess water from the body that normal functioning kidneys would otherwise remove. Dialysis treatment for replacement of kidney functions is critical to many people because the treatment is lifesaving.

One type of kidney failure therapy is Hemodialysis (“HD”), which in general uses diffusion to remove waste products from a patient's blood. A diffusive gradient occurs across the semi-permeable dialyzer between the blood and an electrolyte solution called dialysate or dialysis fluid to cause diffusion.

Hemofiltration (“HF”) is an alternative renal replacement therapy that relies on a convective transport of toxins from the patient's blood. HF is accomplished by adding substitution or replacement fluid to the extracorporeal circuit during treatment. The substitution fluid and the fluid accumulated by the patient in between treatments is ultrafiltered over the course of the HF treatment, providing a convective transport mechanism that is particularly beneficial in removing middle and large molecules.

Hemodiafiltration (“HDF”) is a treatment modality that combines convective and diffusive clearances. HDF uses dialysis fluid flowing through a dialyzer, similar to standard hemodialysis, to provide diffusive clearance. In addition, substitution solution is provided directly to the extracorporeal circuit, providing convective clearance.

Most HD, HF, and HDF treatments occur in centers. A trend towards home hemodialysis (“HHD”) exists today in part because HHD can be performed daily, offering therapeutic benefits over in-center hemodialysis treatments, which occur typically bi- or tri-weekly. Studies have shown that more frequent treatments remove more toxins and waste products and render less interdialytic fluid overload than a patient receiving less frequent but perhaps longer treatments. A patient receiving more frequent treatments does not experience as much of a down cycle (swings in fluids and toxins) as does an in-center patient, who has built-up two or three days' worth of toxins prior to a treatment. In certain areas, the closest dialysis center can be many miles from the patient's home, causing door-to-door treatment time to consume a large portion of the day. Treatments in centers close to the patient's home may also consume a large portion of the patient's day. HHD can take place overnight or during the day while the patient relaxes, works or is otherwise productive.

Another type of kidney failure therapy is peritoneal dialysis (“PD”), which infuses a dialysis solution, also called dialysis fluid or PD fluid, into a patient's peritoneal chamber via a catheter. The PD fluid comes into contact with the peritoneal membrane in the patient's peritoneal chamber. Waste, toxins and excess water pass from the patient's bloodstream, through the capillaries in the peritoneal membrane, and into the PD fluid due to diffusion and osmosis, i.e., an osmotic gradient occurs across the membrane. An osmotic agent in the PD fluid provides the osmotic gradient. Used PD fluid is drained from the patient, removing waste, toxins and excess water from the patient. This cycle is repeated, e.g., multiple times.

There are various types of peritoneal dialysis therapies, including continuous ambulatory peritoneal dialysis (“CAPD”), automated peritoneal dialysis (“APD”), tidal flow dialysis and continuous flow peritoneal dialysis (“CFPD”). CAPD is a manual dialysis treatment. Here, the patient manually connects an implanted catheter to a drain to allow used PD fluid to drain from the patient's peritoneal cavity. The patient then switches fluid communication so that the patient catheter communicates with a bag of fresh PD fluid to infuse the fresh PD fluid through the catheter and into the patient. The patient disconnects the catheter from the fresh PD fluid bag and allows the PD fluid to dwell within the patient's peritoneal cavity, wherein the transfer of waste, toxins and excess water takes place. After a dwell period, the patient repeats the manual dialysis procedure, for example, four times per day. Manual peritoneal dialysis requires a significant amount of time and effort from the patient, leaving ample room for improvement.

APD is similar to CAPD in that the dialysis treatment includes drain, fill and dwell cycles. APD machines, however, perform the cycles automatically, typically while the patient sleeps. APD machines free patients from having to manually perform the treatment cycles and from having to transport supplies during the day. APD machines connect fluidly to an implanted catheter, to a source or bag of fresh PD fluid and to a fluid drain. APD machines pump fresh PD fluid from a dialysis fluid source, through the catheter and into the patient's peritoneal chamber. APD machines also allow for the PD fluid to dwell within the chamber and for the transfer of waste, toxins and excess water to take place. The source may include multiple liters of dialysis fluid, including several solution bags.

APD machines pump used PD fluid from the patient's peritoneal cavity, though the catheter, to drain. As with the manual process, several drain, fill and dwell cycles occur during dialysis. A “last fill” may occur at the end of the APD treatment. The last fill fluid may remain in the peritoneal chamber of the patient until the start of the next treatment, or may be manually emptied at some point during the day.

PD fluid needs to be sterile or very near sterile because it is injected into the patient's peritoneal cavity, and is accordingly considered a drug. While bagged PD fluid is typically properly sterilized for treatment, PD fluid made online or PD machines or cyclers that employ disinfection may need additional sterilization.

There is accordingly a need for an effective, low cost way of providing additional sterilization to fresh PD fluid before it is delivered to a patient.

### SUMMARY

The present disclosure provides a peritoneal dialysis (“PD”) system having a PD machine or cycler that pumps fresh PD fluid through a patient line to a patient and removes used PD fluid from the patient via the patient line. The patient line may be reusable or disposable and in either case operates with and fluidly communicates with a filter set. If the patient line is reusable, the reusable patient line is connected to the filter set at the time of treatment. If the patient line is disposable, the filter set is merged into the disposable patient line in one embodiment. In either configuration a distal end of the filter set may be connected to the patient's transfer set, which in turn communicates fluidly with the patient's indwelling catheter.

The PD machine or cycler may include a durable PD fluid pump that pumps PD fluid through the pump itself without using a disposable component, or a disposable type PD fluid pump including a pump actuator that actuates a disposable, fluid-contacting pumping component, such as a peristaltic pump tube or a flexible pumping chamber. The PD machine or cycler also includes a plurality of valves, which may likewise be flow-through and durable without operating with a disposable component, or be disposable type valves having valve actuators that actuate a disposable, fluid-contacting valve component, such as a tube segment or a cassette-based valve seat.

The pumps and valves are under the automatic control of a control unit provided by the machine or cycler. In an embodiment, the valves include a fresh PD fluid valve that the control unit opens to allow the PD fluid pump to pump fresh PD fluid through a fresh PD fluid lumen of a dual lumen patient line to the patient. The valves also include a used PD fluid valve that the control unit opens to allow the PD fluid pump to pump used PD fluid from the patient through a used PD fluid lumen of the dual lumen patient line. It should be appreciated that while a single PD fluid pump may be used, dedicated fresh and used PD fluid pumps may be used alternatively. Also, a single PD fluid pump may include multiple pumping chambers for more continuous PD fluid flow.

The fresh and used PD fluid lumens may again be reusable or disposable. In the instance in which the fresh and used PD fluid lumens are reusable, the lumens terminate with a patient line connector that connects to a lumen-side connector of the filter set. The lumen-side connector in one embodiment includes a fresh PD fluid port for communication with the fresh PD fluid lumen of the dual lumen patient line and a used PD fluid port for communicating with the used PD fluid lumen of the dual lumen patient line. The lumen-side connector may also include a threaded shroud for threadingly engaging mating threads of the patient line connector. The threading of the patient line connector to the lumen-side connector seals mating ports of the patient line connector to the fresh and used PD fluid ports of the lumen-side connector in one embodiment, e.g., via one or more gasket.

A fresh PD fluid passageway extends from the fresh PD fluid port of the lumen-side connector to a wall, e.g., circular, forming part of the filter housing. The wall forms a fresh PD fluid inlet to a tubular filter membrane. Fresh PD fluid flows through the fresh PD fluid inlet into the interior of the tubular filter membrane in one embodiment. The fresh PD fluid is pressurized within the interior of the tubular filter membrane, forcing the fresh PD fluid to be further filtered through the tubular filter membrane. The tubular filter membrane may be a sterilizing grade or bacteria reduction hydrophilic membrane, which may be formed with porous walls having a pore size of about 0.2 micron through which the fresh PD fluid flows for further filtration. The tubular filter membrane is sized to provide sufficient filtration over multiple patient fills while being small enough not to present discomfort to the patient who is likely sleeping during treatment.

The final filtered fresh PD fluid flows from the interior of the tubular filter membrane to a filtered fluid compartment, e.g., cylindrically shaped, located between the outside surface of the tubular filter membrane and an inside of the filter housing. The final filtered fresh PD fluid flows through a transfer set-side port (common to both fresh and used PD fluid), and into the patient's transfer set, either directly or via a short, flexible tube located between the filter housing and the patient's transfer set. The transfer set-side port may be surrounded by a threaded shroud forming a transfer set-side connector, which either connects directly to a mating connector of the patient's transfer set or to a mating connector of the short tube placed between the filter housing and the patient's transfer set. If the shroud is not provided, the transfer set-side connector may alternatively simply be the transfer set-side port to which the short tube extends over or into for ultrasonically, heat and/or adhesively (e.g., solvent) sealing to the port.

Used PD fluid removed from the patient under negative pressure through the patient's transfer set enters the filter housing through the transfer set-side port of the transfer set-side connector. The used PD fluid enters the filtered fluid compartment within the filter housing and travels from the filtered fluid compartment through a used PD fluid outlet formed in the wall of the filter housing. The used PD fluid exiting the PD fluid outlet flows through a used PD fluid passageway that extends to the used PD fluid port of the lumen-side connector. The used PD fluid exiting the used PD fluid port of the lumen-side connector flows under negative pressure via the PD fluid pump through the used PD fluid lumen of the dual lumen patient line, back to the PD machine or cycler. The PD machine or cycler pumps the used PD fluid under positive pressure to drain.

Used PD fluid does contact the outside surface of the tubular filter membrane but does so in a tangential manner, wherein fibrin, proteins and other particulates within the patient's effluent do not tend to be trapped by or caught on the filter membrane. The filter membrane accordingly remains viable over the course of multiple fills of a PD treatment prior to being discarded with the filter set.

Additionally, the hydrophilic nature of the filter membrane prevents air from migrating across the membrane once the membrane is fully wetted with fresh PD fluid and thus serves a secondary final stage air removal purpose. If needed, however, it is contemplated to provide one or more hydrophobic membrane upstream of the filter membrane (from a fresh PD fluid standpoint), e.g., along circular sides bounding the tubular filter membrane. The one or more hydrophobic membrane allows air to be vented to atmosphere prior to the fresh PD fluid flowing through the filter membrane, e.g., via one or more vent hole provided in one or more wall located adjacent to the one or more hydrophobic membrane.

In light of the disclosure set forth herein, and without limiting the disclosure in any way, in a first aspect of the present disclosure, which may be combined with any other aspect described herein, or portion thereof, a peritoneal dialysis (“PD”) system includes a PD machine: a patient line extending from the PD machine; and a filter set including a filter housing having a tubular filter membrane positioned and arranged to filter fresh PD fluid flowing radially across the tubular filter membrane, and a transfer set-side port positioned and arranged to receive (i) filtered fresh PD fluid during a patient fill and (ii) used PD fluid during a patient drain.

In a second aspect of the present disclosure, which may be combined with any other aspect described herein, or portion thereof, the patient line is a dual lumen patient line including a fresh PD fluid lumen placed in fluid communication with a fresh PD fluid passageway of the filter set, the dual lumen patient line further including a used PD fluid lumen placed in fluid communication with a used PD fluid passageway of the filter set, the fresh PD fluid passageway positioned and arranged to deliver fresh PD fluid to the tubular filter membrane.

In a third aspect of the present disclosure, which may be combined with any other aspect described herein, or portion thereof, the fresh PD fluid passageway is positioned and arranged to deliver fresh PD fluid to an interior of the tubular filter membrane.

In a fourth aspect of the present disclosure, which may be combined with any other aspect described herein, or portion thereof, the PD system includes a wall forming a part of the filter housing, the wall including an inlet to the tubular filter membrane, the inlet in fluid communication with the fresh PD fluid passageway.

In a fifth aspect of the present disclosure, which may be combined with any other aspect described herein, or portion thereof, the wall includes an outlet through which used PD fluid flows to the used PD fluid passageway.

In a sixth aspect of the present disclosure, which may be combined with any other aspect described herein, or portion thereof, the filter housing is configured such that used PD fluid flows across the tubular filter membrane to the outlet.

In a seventh aspect of the present disclosure, which may be combined with any other aspect described herein, or portion thereof, the filter housing is configured such that filtered fresh PD fluid flows into a filtered fluid compartment, the filtered fluid compartment in fluid communication with the transfer set-side port and a used PD fluid outlet, and wherein the PD machine is configured to close a used PD fluid valve during a patient fill, urging the filtered fresh PD fluid to flow to the transfer set-side port.

In an eighth aspect of the present disclosure, which may be combined with any other aspect described herein, or portion thereof, the PD machine is configured to close a fresh PD fluid valve during a patient drain, urging used PD fluid to flow to the used PD fluid outlet.

In a ninth aspect of the present disclosure, which may be combined with any other aspect described herein, or portion thereof, the PD system includes at least one hydrophobic membrane positioned and arranged to vent air from the fresh PD fluid upstream from the tubular filter membrane.

In a tenth aspect of the present disclosure, which may be combined with any other aspect described herein, or portion thereof, the at least one hydrophobic membrane is positioned at at least one end of the tubular filter membrane.

In an eleventh aspect of the present disclosure, which may be combined with any other aspect described herein, or portion thereof, the filter set is configured such that fresh PD fluid is filtered through the tubular filter membrane from the inside-out.

In a twelfth aspect of the present disclosure, which may be combined with any other aspect described herein, or portion thereof, the filter set includes a lumen-side connector and a gasket for sealing between the lumen-side connector and a patient line connector.

In a thirteenth aspect of the present disclosure, which may be combined with any other aspect described herein, or portion thereof, the filter set is configured to connect directly to a patient's transfer set, or wherein the filter set includes a flexible tube configured to connect to the patient's transfer set.

In a fourteenth aspect of the present disclosure, which may be combined with any other aspect described herein, or portion thereof, the PD machine includes a pressure sensor positioned and arranged to sense the pressure of filtered fresh PD fluid downstream from the filter membrane during a patient fill.

In a fifteenth aspect of the present disclosure, which may be combined with any other aspect described herein, or portion thereof, the tubular filter membrane is a sterilizing grade filter membrane or a bacteria reduction filter membrane.

In a sixteenth aspect of the present disclosure, which may be combined with any other aspect described herein, or portion thereof, a filter set for connecting to a patient line includes a filter housing including a tubular filter membrane positioned and arranged to filter fresh PD fluid flowing radially across the tubular filter membrane; a filtered fluid compartment for receiving filtered fresh PD from the tubular filter membrane; and a transfer set-side port positioned and arranged to receive (i) filtered fresh PD fluid from the filtered fluid compartment during a patient fill and (ii) used PD fluid during a patient drain.

In a seventeenth aspect of the present disclosure, which may be combined with any other aspect described herein, or portion thereof, any of the features, functionality and alternatives described in connection with any one or more of FIGS. 1 to 3 may be combined with any of the features, functionality and alternatives described in connection with any other of FIGS. 1 to 3.

In light of the above aspects and the present disclosure herein, it is an advantage of the present disclosure to provide a filter set that operates with a dual lumen patient line.

It is another advantage of the present disclosure to provide a filter set that filters fresh PD fluid and allows used PD fluid to pass without clogging.

It is a further advantage of the present disclosure to provide a filter set having a filtration capacity that is readily adjustable by varying the size of the filter membrane sheet.

It is yet another advantage of the present disclosure to provide a filter set having a venting function that is readily manufactured and that functions regardless of filter orientation.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a schematic view of one embodiment for a peritoneal dialysis system including a patient line filter set having a tubular filter membrane of the present disclosure.

FIG. 2 is a perspective view of the patient line filter set of FIG. 1 during a patient fill.

FIG. 3 is a perspective view of the patient line filter set of FIG. 1 during a patient drain.

### DETAILED DESCRIPTION

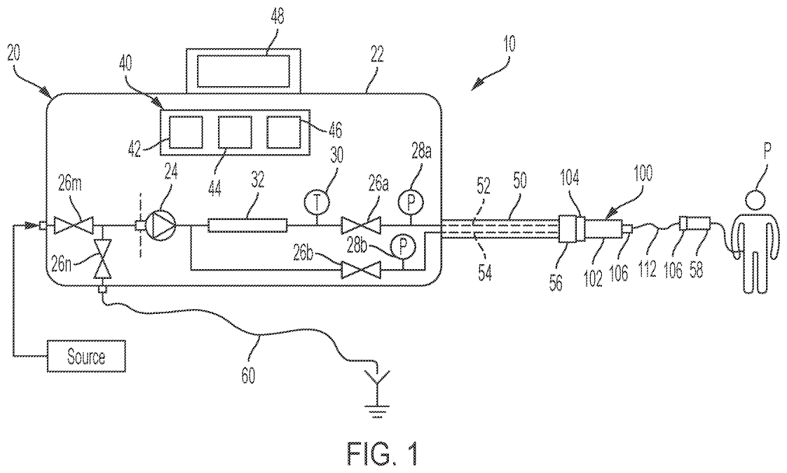

Referring now to the drawings and in particular to FIG. 1, a peritoneal dialysis (“PD”) system 10 is illustrated. PD system 10 includes a PD machine or cycler 20 that pumps fresh PD fluid through a patient line 50 to a patient P and removes used PD fluid from patient P via patient line 50. Patient line 50 may be reusable or disposable and in either case operates with and fluidly communicates with a filter set 100. If patient line 50 is reusable, the reusable patient line is connected to filter set 100 at the time of treatment. If patient line 50 is instead disposable, filter set 100 is merged into or formed with disposable patient line 50 in one embodiment. In either configuration, a distal end of filter set 100 may be connected to the patient's transfer set 58, which in turn communicates fluidly with the indwelling catheter of patient P.

PD machine or cycler 20 may include a housing 22 providing a durable PD fluid pump 24 that pumps PD fluid through the pump itself without using a disposable component. Examples of durable pumps that may be used for PD fluid pump 24 include piston pumps, gear pumps and centrifugal pumps. Certain durable pumps, such as piston pumps are inherently accurate, so that machine or cycler 20 does not require additional volumetric control components. Other durable pumps, such as gear pumps and centrifugal pumps may not be as accurate, such that machine or cycler 20 provides a volumetric control device such as one or more flowmeter (not illustrated).

Pump 24 may alternatively be a disposable type PD fluid pump, which includes a pump actuator that actuates a disposable, fluid-contacting pumping component, such as a peristaltic pump tube or a flexible pumping chamber. Examples of disposable PD fluid pumps that may be used for PD fluid pump 24 include rotary or linear peristaltic pump actuators that actuate tubing, pneumatic pump actuators that actuate cassette sheeting, electromechanical pump actuators that actuate cassette sheeting and platen pump actuators that actuate tubing. It should be appreciated that while a single PD fluid pump 24 may be used, dedicated fresh and used PD fluid pumps may be used alternatively. Also, single PD fluid pump 24 may include multiple pumping chambers for more continuous PD fluid flow.

PD machine or cycler 20 also includes a plurality of valves 26a, 26b, 26m. 26n, which may likewise be flow-through and durable without operating with a disposable component, or be disposable type valves having valve actuators that actuate a disposable, fluid-contacting valve component, such as a tube segment or a cassette-based valve seat. Examples of durable valves that may be used for valves 26a, 26b, 26m. 26n include flow-through solenoid valves. Such valves may be two-way or three-way valves. Examples of disposable valves that may be used for valves 26a, 26b, 26m, 26n include solenoid pinch valves that pinch closed flexible tubing, pneumatic valve actuators that actuate cassette sheeting, and electromechanical valve actuators that actuate cassette sheeting.

Machine or cycler 20 likely includes many valves 26a to 26n. For ease of illustration, machine or cycler 20 is shown having a fresh PD fluid valve 26a that is controlled to open to allow PD fluid pump 24 to pump fresh PD fluid under positive pressure through a fresh PD fluid lumen 52 of dual lumen patient line 50 to patient P. The valves also include a used PD fluid valve 26b that is controlled to open to allow PD fluid pump 24 to pull used PD fluid from patient P under negative pressure through a used PD fluid lumen 54 of dual lumen patient line 50. One or more supply valve 26m is provided to allow selective access to one or more PD fluid source, while valve 26n is provided to allow selective access to a drain via a drain line 60, such as a drain container or house drain.

Machine or cycler 20 in the illustrated embodiment also includes pressure sensors, such as pressure sensors 28a. 28b. Pressure sensor 28a is located just downstream from fresh PD fluid valve 26a, while pressure sensor 28b is located just upstream from used PD fluid valve 26b. Pressure sensor 28a may accordingly sense the pressure in fresh PD fluid lumen 52 of dual lumen patient line 50 even if fresh PD fluid valve 26a is closed, while pressure sensor 28b may sense the pressure in used PD fluid lumen 54 of dual lumen patient line 50 even if used PD fluid valve 26b is closed. Additionally, pressure sensor 28a is positioned to sense the pressure of fresh PD fluid upstream from the filter membrane discussed herein during a patient fill. Pressure sensor 28b perhaps more importantly is positioned to sense the pressure of fresh PD fluid downstream from the tubular filter membrane during a patient fill, which accordingly takes into account any pressure drop across the tubular filter membrane, and which more accurately reflects the pressure at which the PD fluid is being delivered to the patient.

Pump 24 and valves 26a. 26b in the illustrated embodiment are under the automatic control of control unit 40 provided by machine or cycler 20 of system 10, while pressure sensors 28a. 28b (and other sensors) output to control unit 40. Control unit 40 in the illustrated embodiment includes one or more processor 42, one or more memory 44 and a video controller 46. Control unit 40 receives, stores and processes signals or outputs from pressure sensors 28a, 28b, and other sensors provided by machine or cycler 20, such as one or more temperature sensor 30 and one or more conductivity sensor (not illustrated). Control unit 40 may use pressure feedback from one or more of pressure sensor 28a, 28b to control PD fluid pump 24 to pump dialysis fluid at a desired pressure or within a safe pressure limit (e.g., within 0.21 bar (three psig) of positive pressure to a patient's peritoneal cavity and −0.10 bar (−1.5 psig) of negative pressure from the patient's peritoneal cavity).

Control unit 40 uses temperature feedback from one or more temperature sensor 30 for example to control a heater 32, such as an inline heater, to heat fresh PD fluid to a desired temperature, e.g., body temperature or 37° C. In one embodiment, heater 32 is used additionally to heat a disinfection fluid, such as fresh PD fluid, to disinfect PD fluid pump 24, valves 26a to 26n, heater 32 and all reusable fluid lines within machine or cycler 20 to ready the machine or cycler for a next treatment. The additional filtration discussed herein provides a layer of protection in addition to the heated fluid disinfection to ensure that the PD fluid is safe for delivery to patient P.

Video controller 46 of control unit 40 interfaces with a user interface 48 of machine or cycler 20, which may include a display screen operating with a touchscreen and/or one or more electromechanical button, such as a membrane switch. User interface 48 may also include one or more speaker for outputting alarms, alerts and/or voice guidance commands. User interface 48 may be provided with the machine or cycler 20 as illustrated in FIG. 1 and/or be a remote user interface operating with control unit 40. Control unit 40 may also include a transceiver (not illustrated) and a wired or wireless connection to a network, e.g., the internet, for sending PD treatment data to and receiving prescription instructions from a doctor's or clinician's server interfacing with a doctor's or clinician's computer.

Referring to FIGS. 1 to 3, as mentioned above, fresh and used PD fluid lumens 52 and 54 of dual lumen patient line 50 may again be reusable or disposable. In the instance in which dual lumen patient line 50 is reusable, the lumens terminate with a connector 56 that connects to a lumen-side connector 104 of filter set 100. Lumen-side connector 104 in one embodiment includes a fresh PD fluid port 104a for communication with fresh PD fluid lumen 52 of dual lumen patient line 50 and a used PD fluid port 104b for communicating with used PD fluid lumen 54 of dual lumen patient line 50. Fresh PD fluid port 104a and used PD fluid port 104b in the illustrated embodiment are surrounded by a shroud 104s of lumen-side connector 104, wherein shroud 104s is formed with threads 104c for threadingly engaging mating threads of patient line connector 56. The threading of patient line connector 56 to lumen-side connector 104 seals mating ports (not illustrated) of patient line connector 56 to fresh and used PD fluid ports 104a and 104b of the lumen-side connector 104 in one embodiment, e.g., via one or more compressible gasket (not illustrated), such as a silicone or other suitable rubber gasket. In the illustrated embodiment, the front of shroud 104s is formed with a keyed opening 104k. Patient line connector 56 is formed with a mating key so that the patient line connector can only be introduced into shroud 104s in a proper orientation, aligning fresh PD fluid lumen 52 with fresh PD fluid port 104a and used PD fluid lumen 54 with used PD fluid port 104b.

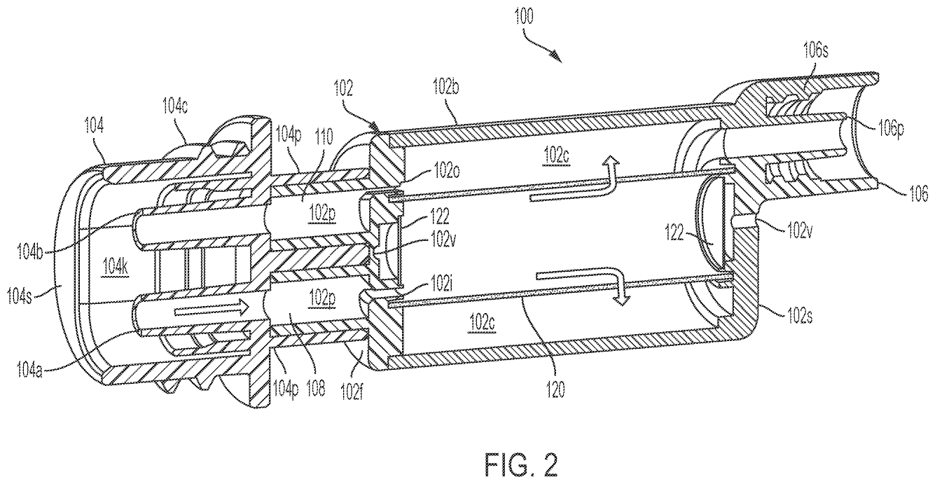

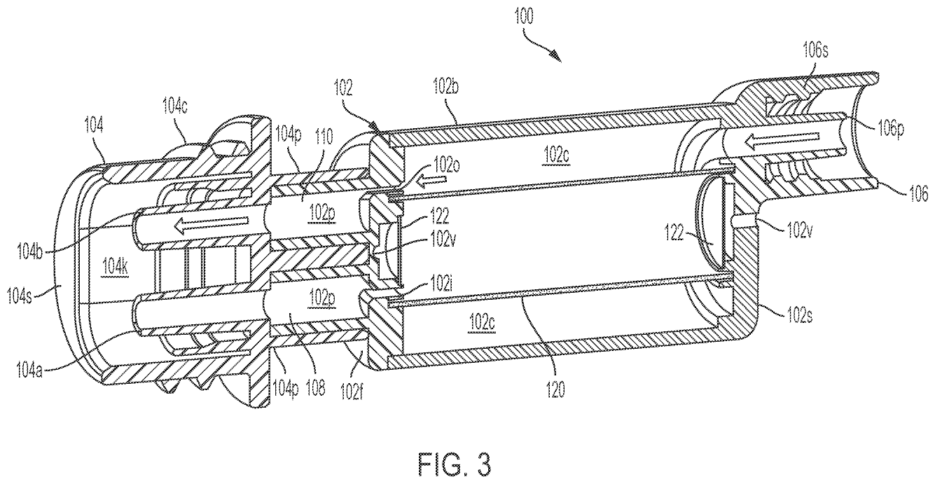

FIGS. 2 and 3 illustrate that a fresh PD fluid passageway 108 extends from fresh PD fluid port 104a of lumen-side connector 104 to a first wall 102f forming a part of a filter housing 102. First wall 102f in the illustrated embodiment forms or defines a fresh PD fluid inlet 102i to the interior of a tubular filter membrane 120. First wall 102f also forms ports 102p that are ultrasonically, heat and/or adhesively (e.g., solvent) sealed to mating ports 104p of lumen-side connector 104 in the illustrated embodiment.

Fresh PD fluid in FIG. 2 flows along fresh PD fluid passageway 108 during a patient fill, as illustrated by a flow direction arrow, through fresh PD fluid inlet 102i and into the interior of tubular filter membrane 120 in one embodiment. The fresh PD fluid is pressurized within the interior of the tubular filter membrane 120, forcing the fresh PD fluid to be further filtered through the tubular filter membrane. Tubular filter membrane 120 may be a sterilizing grade or bacteria reduction hydrophilic membrane, which may be formed with porous walls having a pore size of about 0.2 micron through which the fresh PD fluid flows for further filtration. Tubular filter membrane 120 may be made of, for example, polysulfone or polyethersulfone blended with polyvinylpyrrolidone. Tubular filter membrane 120 is sized, e.g., (i) 30 to 40, such as 35, millimeters (“mm”) in length, (ii) 8 to 22, such as 10, mm in diameter, and (iii) 10 to 20 centimeters² (“cm²”) in surface area, to provide sufficient filtration over multiple patient fills while being small enough not to present discomfort to the patient who is likely sleeping during treatment.

The final filtered fresh PD fluid in FIG. 2 flows from the interior and across tubular filter membrane 120, as illustrated by flow direction arrows, to a filtered fluid compartment 102c. e.g., cylindrically shaped, located between the outside surface of tubular filter membrane 120 and an inside of a body 102b, e.g., cylindrical body, of filter housing 102. The final filtered fresh PD fluid during the patient fill flows through a transfer set-side port 106p (common to both fresh and used PD fluid), and into the patient's transfer set 58, either directly or via a short, flexible tube 112 located between filter housing 102 and transfer set 58 of patient P (FIG. 1). The transfer set-side port 106p may be surrounded by a threaded shroud 106s forming a transfer set-side connector 106, which either connects directly to a mating connector of transfer set 58 of patient P or to a mating connector of short tube 112 placed between filter housing 102 and the patient's transfer set 58. If shroud 106s is not provided, transfer set-side connector 106 may alternatively simply include transfer set-side port 106p to which short tube 112 extends over or into for ultrasonically, heat and/or adhesively (e.g., solvent) sealing to transfer set-side port 106p. Likewise, if dual lumen patient line 50 is disposable, lumen-side connector 104 may alternatively simply include ports. e.g., fresh and used PD fluid ports 104a and 104b, to which fresh and used PD fluid lumens 52 and 54 respectively extend over or into for like sealing to the ports.

FIGS. 2 and 3 illustrate that a second wall 102s of filter housing 102 may be molded with body 102b and transfer set-side connector 106. Here, filter set 100 may be assembled, e.g., ultrasonically sealed, heat sealed and/or solvent bonded using three molded pieces, namely. (i) lumen-side connector 104, (ii) first wall 102f, and (iii) housing 102/transfer set-side connector 106. Filter set 100 may also include short, flexible tube 112 as discussed herein. Any of the molded pieces may be made from any one or more plastic, such as, polystyrene (“PS”), polycarbonate (“PC”), blends of polycarbonate and acrylonitrile-butadiene-styrene (“PC/ABS”), polyvinyl chloride (“PVC”), polyethylene (“PE”), polypropylene (“PP”), polyesters like polyethylene terephthalate (“PET”), or polyurethane (“PU”). Flexible tube 112 may be made of PVC or non-PVC flexible tubing.

FIGS. 1 and 3 illustrate that used PD fluid removed from patient P under negative pressure during a patient drain via transfer set 58 enters filter housing 102 through transfer set-side port 106p of transfer set-side connector 106, as illustrated by a flow direction arrow: Used PD fluid enters filtered fluid compartment 102c located within filter housing 102 and travels from filtered fluid compartment 102c through a used PD fluid outlet 102o, as illustrated by a flow direction arrow; formed in first wall 102f. Used PD fluid exiting PD fluid outlet 102o flows through a used PD fluid passageway 110 that extends to used PD fluid port 104b of lumen-side connector 104. Used PD fluid exiting used PD fluid port 104b of the lumen-side connector 104, as illustrated by a flow direction arrow, flows under negative pressure via PD fluid pump 24 through used PD fluid lumen 54 of dual lumen patient line 50, back to PD machine or cycler 20. PD machine or cycler 20 pumps the used PD fluid under positive pressure to drain via drain line 60.

Used PD fluid does contact the outside surface of tubular filter membrane 120 but does so in a tangential manner, wherein fibrin, proteins and other particulates within the patient's effluent do not tend to be trapped by or caught on the filter membrane. Tubular filter membrane 120 accordingly remains viable over the course of multiple fills of a PD treatment prior to being discarded with filter set 100.

Additionally, the hydrophilic nature of tubular filter membrane 120 prevents air from migrating across the membrane once membrane 120 is fully wetted with fresh PD fluid and thus serves a secondary final stage air removal purpose. If needed, however, it is contemplated to provide one or more hydrophobic membrane 122 upstream of tubular filter membrane 120 (from a fresh PD fluid standpoint), e.g., along one or more circular side bounding tubular filter membrane 120. One or more hydrophobic membrane 122 may be constructed for example from polytetrafluoroethylene (“PTFE”). The one or more hydrophobic membrane 122 in the illustrated embodiment is ultrasonically sealed, heat sealed and/or solvent bonded to a cylindrical mount extending from one or more first and/or second wall 102f, 102s of filter housing 102. One or more hydrophobic membrane 122 allows air to be vented to atmosphere prior to the fresh PD fluid flowing through tubular filter membrane 120, e.g., via one or more vent hole 102v provided in one or more first and/or second wall 102f, 102s located adjacent to one or more hydrophobic membrane 122.

Referring to FIGS. 1 and 2, during a patient fill, control unit 40 causes used PD fluid valve 26b to be closed. Also during the patient fill, used PD fluid lumen 54 and used PD fluid passageway 110 are filled with fresh and/or used PD fluid. The result is that filtered fresh PD fluid entering filtered fluid compartment 102c through the pores of tubular filter membrane 120 is effectively prevented from flowing through PD fluid outlet 102o into used PD fluid lumen 54. Fresh PD fluid is therefore delivered from filter set 100 to patient P.

Referring to FIGS. 1 and 3, during a patient drain, control unit 40 causes fresh PD fluid valve 26a to be closed. Also during the patient drain, fresh PD fluid lumen 52, fresh PD fluid passageway 108 and the interior of tubular filter membrane 120 are filled with fresh PD fluid. The result is that used PD fluid entering filtered fluid compartment 102c from transfer set-side port 106p is effectively prevented from flowing through the pores of tubular filter membrane 120. Used PD fluid is therefore delivered from filter set 100 to used PD fluid lumen 54.

It should be understood that various changes and modifications to the presently preferred embodiments described herein will be apparent to those skilled in the art. It is therefore intended that any or all of such changes and modifications may be covered by the appended claims. For example, while a dual lumen patient line 50 is shown operating with fresh and used PD fluid ports 104a and 104b of lumen-side connector 104, the patient line may alternatively be a single lumen patient line, which communicates with a single port within lumen-side connector 104. Here, check valves may be sealed and oriented within the fresh and used PD fluid passageways 108, 110 to direct fresh and used PD fluid as needed. Additionally, while fresh PD fluid is described as being filtered through the tubular filter membrane 120 from the inside-out, the fresh PD fluid may alternatively be filtered through tubular filter membrane 120 from the outside-in.

The invention is claimed as follows:

1. A peritoneal dialysis (“PD”) system (10) comprising:
a PD machine (20);
a patient line (50) extending from the PD machine (20); and
a filter set (100) including a filter housing (102) having a tubular filter membrane (120) positioned and arranged to filter fresh PD fluid flowing radially across the tubular filter membrane (120), and a transfer set-side port (106p) positioned and arranged to receive (i) filtered fresh PD fluid during a patient fill and (ii) used PD fluid during a patient drain.

2. The PD system (10) according to claim 1, wherein the patient line (50) is a dual lumen patient line including a fresh PD fluid lumen (52) placed in fluid communication with a fresh PD fluid passageway (108) of the filter set (100), the dual lumen patient line (50) further including a used PD fluid lumen (54) placed in fluid communication with a used PD fluid passageway (110) of the filter set (100), the fresh PD fluid passageway (108) positioned and arranged to deliver fresh PD fluid to the tubular filter membrane (120).

3. The PD system (10) according to claim 2, wherein the fresh PD fluid passageway (108) is positioned and arranged to deliver fresh PD fluid to an interior of the tubular filter membrane (120).

4. The PD system (10) according to claim 2, which includes a wall (102f) forming a part of the filter housing (102), the wall (102f) including an inlet (102i) to the tubular filter membrane (120), the inlet (102i) in fluid communication with the fresh PD fluid passageway (108).

5. The PD system (10) according to claim 4, wherein the wall (102f) includes an outlet (102o) through which used PD fluid flows to the used PD fluid passageway (110).

6. The PD system (10) according to claim 5, wherein the filter housing (102) is configured such that used PD fluid flows across the tubular filter membrane (120) to the outlet (102o).

7. The PD system (10) according to claim 1, wherein the filter housing (102) is configured such that filtered fresh PD fluid flows into a filtered fluid compartment (102c), the filtered fluid compartment (102c) in fluid communication with the transfer set-side port (106p) and a used PD fluid outlet (102o), and wherein the PD machine (20) is configured to close a used PD fluid valve (26b) during a patient fill, urging the filtered fresh PD fluid to flow to the transfer set-side port (106p).

8. The PD system (10) according to claim 7, wherein the PD machine (20) is configured to close a fresh PD fluid valve (26a) during a patient drain, urging used PD fluid to flow to the used PD fluid outlet (102o).

9. The PD system (10) according to claim 1, which includes at least one hydrophobic membrane (122) positioned and arranged to vent air from the fresh PD fluid upstream from the tubular filter membrane (120).

10. The PD system (10) according to claim 9, wherein the at least one hydrophobic membrane (122) is positioned at at least one end of the tubular filter membrane (120).

11. The PD system (10) according to claim 1, wherein the filter set (100) is configured such that fresh PD fluid is filtered through the tubular filter membrane (120) from inside-out.

12. The PD system (10) according to claim 1, wherein the filter set (100) includes a lumen-side connector (104) and a gasket for sealing between the lumen-side connector (104) and a patient line connector (56).

13. The PD system (10) according to claim 1, wherein the filter set (100) is configured to connect directly to a patient's transfer set, or wherein the filter set (100) includes a flexible tube (112) configured to connect to the patient's transfer set.

14. The PD system (10) according to claim 1, wherein the PD machine (20) includes a pressure sensor (28b) positioned and arranged to sense a pressure of the filtered fresh PD fluid downstream from the tubular filter membrane (112) during a patient fill.

15. The PD system (10) according to claim 1, wherein the tubular filter membrane (120) is a sterilizing grade filter membrane or a bacteria reduction filter membrane.

16. A filter set (100) for connecting to a patient line, the filter set (100) comprising:
a filter housing (102) including a tubular filter membrane (120) positioned and arranged to filter fresh PD fluid flowing radially across the tubular filter membrane (120);
a filtered fluid compartment (102c) for receiving filtered fresh PD from the tubular filter membrane (120); and
a transfer set-side port (106p) positioned and arranged to receive (i) filtered fresh PD fluid from the filtered fluid compartment (102c) during a patient fill and (ii) used PD fluid during a patient drain.
